(19) United States
(12) Patent Application Publication
(10) Pub. No.: US 2026/0071192 A1
(43) Pub. Date: Mar. 12, 2026

(54) METHOD OF DEVELOPING DIVERSE SYNTHETIC PHAGE LIBRARIES TO OVERCOME RESISTANT KLEBSIELLA SPP.

(71) Applicant: United States of America as Represented by the Secretary of the Navy, Silver Spring, MD (US)

(72) Inventors: Jinny L. LIU, Ellicott City, MD (US); Scott DEAN, Bowie, MD (US); Biswajit BISWAS, Germantown, MD (US)

(21) Appl. No.: 19/321,394

(22) Filed: Sep. 8, 2025

Related U.S. Application Data
(60) Provisional application No. 63/692,873, filed on Sep. 10, 2024.

(51) Int. Cl. C12N 7/00; C07K 14/005

(52) U.S. Cl. CPC C12N 7/00; C07K 14/005; C12N 2795/00021; C12N 2795/00022

(57) ABSTRACT

Bacteriophages, also called phage, represent a tool to combat drug resistance in bacteria. Analysis revealed indicated that, for Klebsiella phage, the most important host range determining region is located at C-terminus (last ˜200 amino acids) of tail protein. A machine learning strategy was used to modify tail proteins of Klebsiella phage, thereby generating phages libraries effective in overcoming phage resistance in host bacteria. The technique is expected to be useful to modify other types of phages.

### CROSS-REFERENCE TO RELATED APPLICATIONS

This applications claims the benefit of U.S. Provisional Patent Application No. 63/692,873 filed on Sep. 10, 2024, which is incorporated herein by reference in its entirety.

### FEDERALLY-SPONSORED RESEARCH AND DEVELOPMENT

The United States Government has ownership rights in this invention. Licensing inquiries may be directed to NMRC OPBD, 503 Robert Grant Ave., Silver Spring, MD 20910-7500, or usn.detrick.nmrc.mbx.technology-transfer@health.mil, referencing Navy Case 211872.

### INCORPORATION BY REFERENCE

This Application incorporates by reference the Sequence Listing XML file submitted herewith via the patent office electronic filing system having the file name “211872US2 sequences.xml” and created on Aug. 27, 2025, with a file size of 1,705,299 bytes.

### BACKGROUND

Antibiotic resistance poses a demanding challenge in the treatment of bacterial infection. Synthetic phages tailored with desired properties, such as high potency and altered host ranges, offer a potential solution.

Advances in biology have provided necessary tools and strategies to generate desired synthetic phages. To alter host ranges, recombinant phage genomes with mutations in tail fibers in phages have been constructed in a yeast-based platform and then transformed into E. coli hosts to produce recombinant (also referred as synthetic) phages (Ando et al., 2015). Synthetic phages with smaller genomes of less than about 60 kb have been successfully constructed, however challenges exist in the construction of large phage genomes using yeast-based platforms. The recombination efficiency is usually low and the isolation of yeast artificial chromosome DNA with recombinant phage DNA has lower yield of the recombinant DNA. Another method to prepare synthetic coli phages was to assemble the synthetic phage genomes in vitro in combination with in vivo DNA transduction (Yosef et al., 2017; Kilcher et al., 2018). This approach involves the synthesis of large DNA sequences assembled by Gibson assembly, however, synthesis of large piece DNA fragments, over 10 kb, is difficult and costly. Another approach, based on the tail protein (gp17) crystal structure, was to introduce random mutations into critical loop domains within potential HRDR (host range determining region), subsequently cloned into E. coli plasmids and produced recombinant phages by the infection of parental phages (Yehl et al., 2019). The resulting synthetic coli phages were able to overcome drug resistant E. coli strains.

The above techniques struggle to overcome fast emerging pathogenic bacteria, such as Klebsiella spp. These species, mainly K. pneumoniae (Kp) and K. oxytoca, are major causes of bacterial nosocomial infections, including respiratory tract infections, urinary tract infections, and wound infections during prolonged field care. Such infections contribute to increased mortality and morbidity.

Klebsiella genomes are very flexible and easily recombined with each other to develop resistance to phages and antibiotics, such as carbapenems, by acquiring the ability to resist antibiotics. Their ease of acquiring drug resistance makes them labeled as emerging superbugs, thus rendering critical the development of potent synthetic phages as alternatives to antibiotics.

A need exists to address the limitations of phage therapy and combat the growing challenges of antimicrobial resistance and phage resistance in Klebsiella.

### BRIEF SUMMARY

Described herein are methods of developing synthetic Klebsiella phages, synthetic phage libraries containing diverse tail fiber sequences, and the phage sequences developed by these methods.

In a first embodiment, a method of preparing a synthetic bacteriophage library comprises the steps of providing a selection of bacteriophage sequences, using machine learning and structure prediction modeling to identify a likely receptor binding domain in the selection, then introducing random mutations therein and recombining them into the phage genomes to develop diverse synthetic phages. In further embodiments, the synthetic phages are testing against suitable bacteria to identify phages active against desired targets such as drug-resistant and/or phage-resistant bacteria.

A still further embodiment is a bacteriophage library in a state of having been developed via the method of the first embodiment.

In a phage embodiment, a bacteriophage comprises at least one mutation in a region selected from the group consisting of the BC loop, the EF loop, FG1 loop, and the FG2 loop, wherein the at least one mutation comprises a protein sequence selected from the group consisting of SEQ ID NOs: 19 to 88 in the BC loop, SEQ ID NOs: 90 to 123 in the EF loop, SEQ ID NOs: 125 to 175 in the FG1 loop, and SEQ ID NOs: 177 to 225 in the FG2 loop.

In another variation of the phage embodiment, said at least one mutation comprises two or more mutations in at least two of said loops comprise the mutations. In further variations, three or four of said loops comprise the mutations.

In a further variation of the phage embodiment, bacteriophage comprises a wild-type protein sequence of SEQ ID NO: 227 or SEQ ID NO: 228 prior to taking into account said one or more mutations.

In yet another variation of the phage embodiment, the bacteriophage is capable of infecting Klebsiella.

Still another embodiment is a nucleic acid encoding at least a portion of the C-terminal-most 200 amino acid residues of a phage tail protein sequence, wherein said at least a portion comprises at least one mutation in a region selected from the group consisting of the BC loop, the EF loop, FG1 loop, and the FG2 loop, wherein the at least one mutation comprises a protein sequence selected from the group consisting of SEQ ID NOs: 19 to 88 in the BC loop, SEQ ID NOs: 90 to 123 in the EF loop, SEQ ID NOs: 125 to 175 in the FG1 loop, and SEQ ID NOs: 177 to 225 in the FG2 loop.

Yet another embodiment is a phage library comprising a plurality of bacteriophage of the phage embodiments, wherein the bacteriophage comprise at least two different phages each having different mutations. In one aspect, the library comprises phage of both the Φ115 and Φ100 stock (SEQ ID NOs: 1 and 3, respectively) including mutations thereof as described herein.

Still further embodiments of phages and libraries thereof include phages having protein sequences encoded by SEQ ID NOs: 229 to 225 and minor variations thereof that encompass sequences of 99% or better identity. Additional embodiments are the nucleic acids of SEQ ID NOs: 229 to 225 and variations thereof that encode the same proteins due to codon degeneracy.

In still another embodiment, a method of treating bacterial infection comprises administering at least one phage or library thereof as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C present a confusion matrix displaying the classification performance of gradient boosting models, Klebsiella vs. others, showing results of machine learning analysis for whether an unknown phage tail protein sequence binds to Klebsiella or a different species. The analysis used 143 Klebsiella phages tail fiber sequences and 642 other phage tail fibers. This indicates that the 200 amino acid residue region at the C terminal end of the tail protein (C-200) is a host range determining region (HRDR), as seen in FIG. 1A.

FIG. 2 shows the predicted structure for the C-terminal-most 200 amino acid residues (C-200) of Klebsiella phage tail fiber and identified loop domains in two different orientations.

FIG. 3 depicts an alignment of C-200 tail region showing the loop regions and highlighting areas where the synthetic phage described herein have mutations. The complete sequences of the two phage tail proteins shown, include the C-200 regions, are provided in SEQ ID NOs: 227 and 228.

FIG. 4 provides an electron micrographic image of typical phages obtained by these techniques. Imagery indicate that each phage has an icosahedral capsid (average diameter: 63.86 nm) and a flexible tail (average length: 164.36 nm), characteristic of the Siphoviridae family.

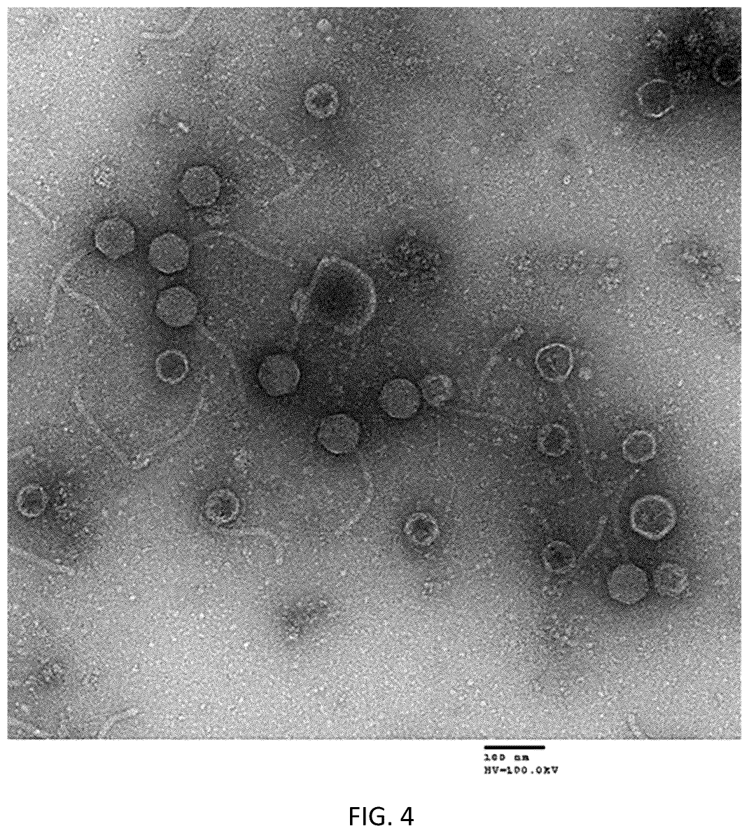

FIGS. 5A and 5b show results of testing of parental phage and synthetic phage library. The chart in FIG. 5A presents measurements of respiration rate of Klebsiella pneumoniae strain Kp294 in the presence of 4 synthetic phages and Kpφ115, Kpφ100 and Kpφ111 (also referred as φ115, φ100, and φ111, respectively). FIG. 5B depicts plates showing lawns of bacterial growth, with spots indicating lysis by the phage being tested at various dilutions. In the top left (position 1a) is the natural Kp294 bacteria with the parental φ115, indicating lysis. Below that in position 1b is the natural Kp bacteria and the synthetic phage library. In each remaining pair (2-6), the top plates (positions 2a-6a), representing five phage resistant strains, Kp294R1, R2, R6, R8 and R9, respectively, that were spotted with parental phage and the bottom plate (positions 2b-6b) those same strains spotted with the synthetic phage.

FIGS. 6A-6C relate to synthetic phage library growth on a panel of multi-drug resistant (MDR) MRSN of Klebsiella pneumoniae. FIG. 6A is a heatmap showing the growth of bacteria from the 96-strain clinical isolates panel. WT indicates φ115-1 stock containing the mixture of φ115 and φ100, while the other 4 are sub-libraries of synthetic phages. FIG. 6B depicts a plate showing a lawn of bacterial growth from strain MRSN 13761, with spots indicating lysis. The initial mixture of φ115 and φ100 showed no lysis at any dilution, and the synthetic phage sub library, FG1-φ115, showed clear lysis spots. FIG. 6C depicts a plate showing a lawn of bacterial growth from strain MRSN 25616. The WT showed no lysis at any dilution, while the sub-synthetic phage library, FG1-φ115 showed clear lysis spots.

FIGS. 7A and 7B offer a host range analysis of individual synthetic phages using a multi-drug resistance panel. FIG. 7A is a chart showing which synthetic phage isolates were able to lyse various strains from the panel. The chart depicts the parental phage strains, φ115 and φ100 and single isolated recombinant phage strains, isolates 1 and 6. FIG. 7B compares the number of strains lysed by the parental phages and selected isolates using both a liquid lysis test and a spot test on a bacterial lawn.

FIGS. 8A-8C relate to burst size measurements in three bacterial strains and four phages. Error bars represent standard deviation and each value was obtained from 2 biological replicate measurements. FIG. 8A presents a comparison of burst size measurements in Kp294 for the parental Φ115 and Φ100 compared to synthetic phage isolates numbers 1 and 6 (p=0.0031 for φ115 vs φ100; p=0.46 for φ115 vs isolate #6; p=0.28 for φ115 vs isolate #1). “*” represents p<0.005 (p=0.0031). FIG. 8B provides burst size measurements in Kp294R1. The value for Φ115 was measured at zero with no lysis observed. The two-sided t-test showed that p=0.45 for φ100 vs isolate #6 and p=54 for φ100 vs isolate #1. FIG. 8C shows provides burst size measurements in Kp294R1, a phage-resistant Kp294 variant. The value for Φ115 was measured at zero with no lysis observed. Two-sided t-test showed that p=0.31 for φ100 vs isolate #6 and p=21 for φ100 vs isolate #1.

FIG. 9 depicts a genomic sequence alignment showing the origin of the DNA in the genome of each synthetic phage isolate, indicating whether the DNA originated from the parental φ100 or φ115 or is unknown. Phage isolates 1, 6, 7 and 8, were isolated on MRSN 13761, while phage isolates 13 and 18 were isolated from Kp294R1. TF is abbreviated for tail fiber. STF stands for short tail fiber, while LTF stands for long tail fiber in φ100 genome. See Table 3 for sequence information.

### DETAILED DESCRIPTION

### Definitions

Before describing the present invention in detail, it is to be understood that the terminology used in the specification is for the purpose of describing particular embodiments and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of 10% of that stated.

### Overview

The development of synthetic Klebsiella phage libraries described herein employed Klebsiella phages and corresponding bacterial hosts. With the aid of machine learning and structure prediction modeling, potential receptor binding domain within host-range-determining regions (HRDRs) in the tail fiber were identified. Then, random mutations were introduced into the binding domains and recombined into the phage genomes to develop diverse synthetic phages.

This method involved a parental phage scaffold, the corresponding host, and the primary sequence of tail fiber for predicting HRDR structure and identifying the potential loops without actual crystal structures. An in-house plasmid was developed for cloning the Klebsiella phage tail fiber gene and selected a Klebsiella pneumoniae host sensitive to kanamycin to produce the synthetic Klebsiella phages.

### Synthetic Phage

First a “chassis phage” was selected. The below examples primarily used Kpφ115, an anti-Klebsiella phage from Naval Medical Research Command with a genomic sequence provided in SEQ ID NO: 1. The genomes of related phage are described SED ID NOs: 2 and 3 (Kpφ111 and Kpφ100, respectively) and can also serve as chassis phage. Indeed, practically any phage could be modified using the techniques described herein.

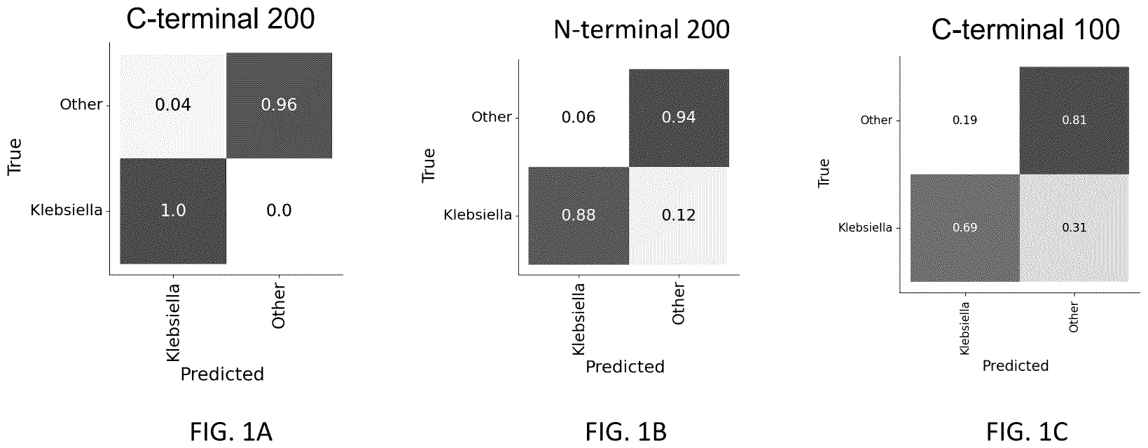

A machine learning model was employed to locate potential receptor binding regions within Klebsiella tail fibers. A gradient boosting model trained in-house was performed on phage tail fibers. The gradient boosting model was implemented using Sci-Kit Learn framework in Python (Pedregosa et al., 2011). The gradient boosting model was ˜98% accurate at distinguishing Klebsiella phage tail proteins versus other bacterial hosts. This model, specifically GradientBoostingClassifie was trained on several different genomes, totaling 785 sequences, of which there were 143 Klebsiella phage tail proteins. FIGS. 1A-1C depict a confusion matrix from this analysis.

The machine learning analysis revealed indicated that the most important HRDR, effectively the phage receptor binding domain (RBD), is located at C-terminus (last ˜200 amino acids) of tail protein of Klebsiella phage as indicated in FIG. 1A. This prediction is somewhat different from the previous study by Yehl et al., who identified via a different method that the C-terminal region (˜100 amino acids) in gp17 altered the host specificity. In the present testing, use of only the C-terminal 100 amino acid provided significantly deteriorated performance (FIG. 1C compared to FIG. 1A), suggesting that Yehl et al. maybe have missed an important section of the tail fiber protein sequence. It is also possible that the discrepancy is from the inherent properties among different bacteria species. Use of in-house gradient boosting model enables very fine-grained highlighting of regions essential for binding and which specific amino acids are the top candidates for mutations, information useful to guide mutation selection to generate phage diversity.

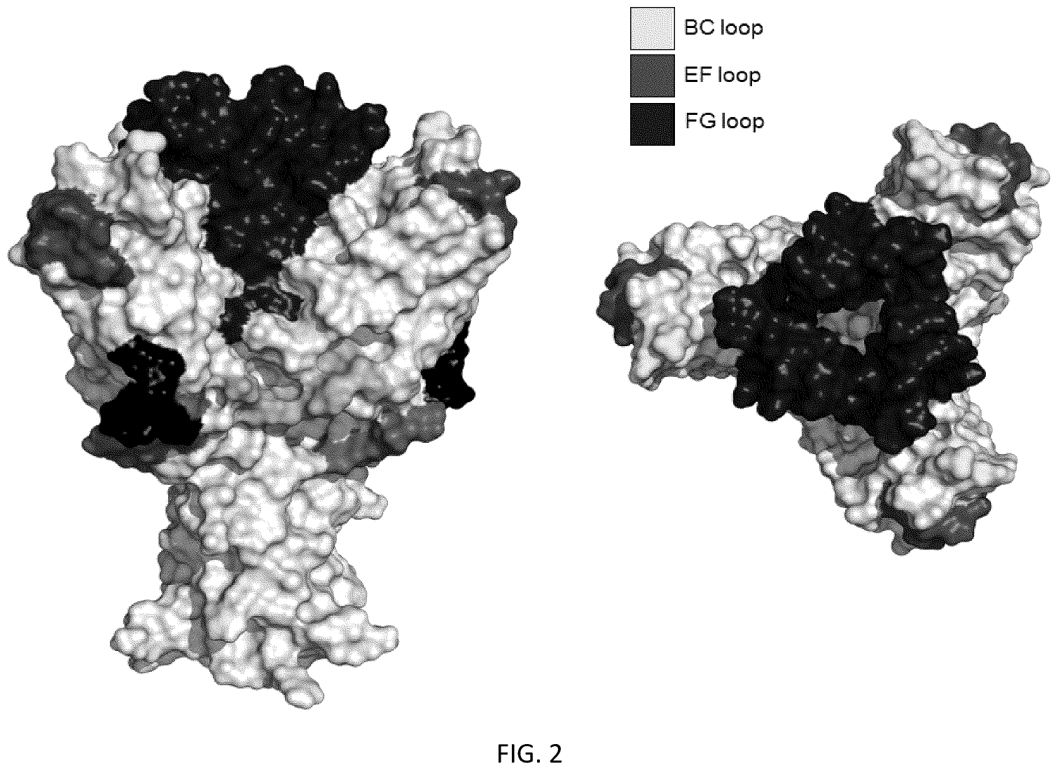

The predicted structure of tail fiber protein shows a barrel for HRDR (FIG. 2). The identified loop domains are exposed on the exterior of the barrel and likely bind to bacterial receptors.

A synthetic phage scaffold derived from Naval Medical Research Command phage Kpφ115 was used for structure prediction of the tail fiber protein. Alphafold and Alphafold 2 were used for predicting the structure and locating the potential loop domains binding to bacterial receptors.

For cloning of phage tail fibers, a new plasmid backbone (PETori-Kan-Ori-f1 DNA fragment, 2085 bp) with Kanamycin resistant markers was constructed by PCR amplification. This involved pet28a as template and primers, pKan115TF F1/pKan115TF Rev. Kp tail fiber (TF) DNA fragments (3771 bp) were amplified using Kpφ115 as template with primers Kpφ115TF F1/Kpφ115 Rev (see Table 1). Amplified pET ori-Kan-f1ori DNA fragments and Kp TF DNA fragments were purified and ligated by Gibson assembly (New England Biolabs). The resulting plasmids, pKp115-19, were then transformed to DH5α/DH10β according to manufacturer's protocol, following plating the bacteria onto LB plus kanamycin (50 μg/mL) agar plates and incubating the plates at 37° C. overnight. The next day, five colonies were selected and grown in 2 mL TB (Terrific broth) mixed with 50 μg/mL kanamycin at 37° C. shaking at 250 rpm overnight. DNA were isolated using miniprep kit (Qiagen) and sent out to sequence using Kpφ115TF seq F1, Kpφ115TF seq F5, Kpφ115TF seq R1/R4/R5.

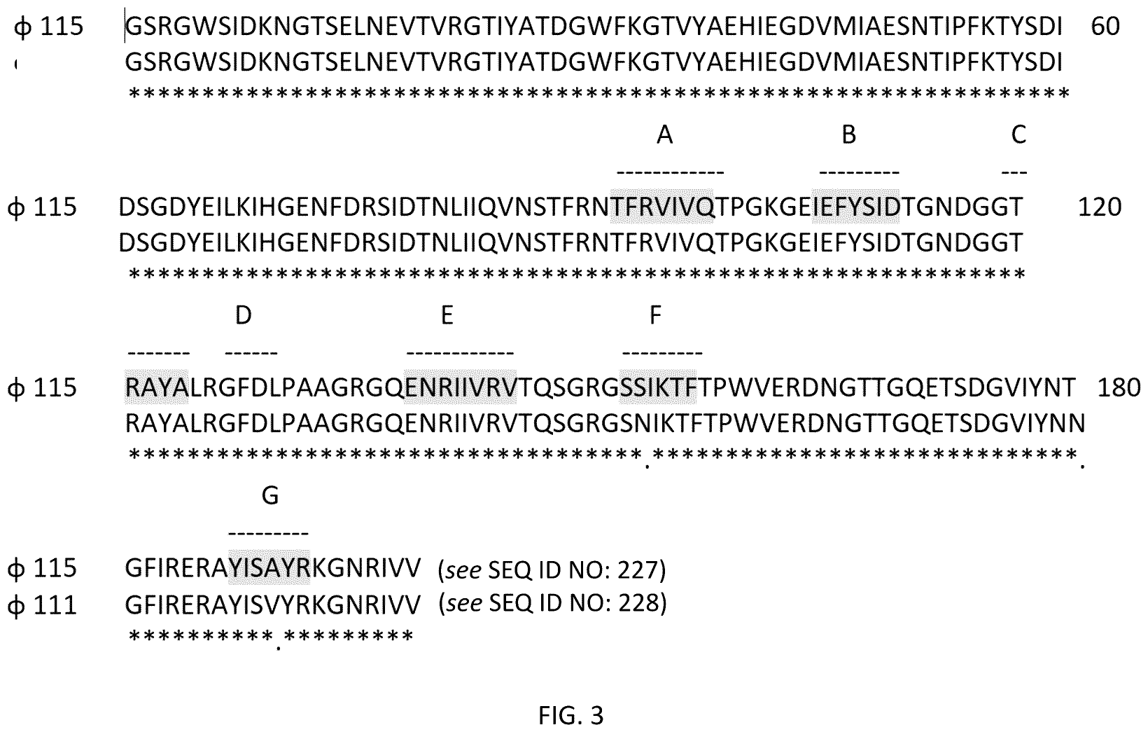

Phage tail variants were generated by introducing random mutations into the predicted loop domain. Klebsiella phage tail variants were amplified using pKp115-19 DNA and 4 primer pairs, Kpφ115TF BClp F1/Kpφ115TF BC lp R1, Kpφ115TF EFlp F1/Kpφ115TF EFlp R1, Kpφ115 FGlp F1/Kpφ115 FGlp R1, and Kpφ115TF FGlp2 F2/Kpφ115TF FGlp2 R2 (listed in Table 1) according to manufacturer's protocol (Q5 DNA polymerase, New England Biolabs). The resulting PCR fragments were then separated on 1% Tris acetate EDTA agarose gel and purified using QIAQUICK gel purification kit followed by QIAQUICK PCR kit (Qiagen Inc). The resulting linear PCR DNA fragments (0.1-0.5 μg) were then re-ligated using HIFI builder (NEB) followed by transformation into competent DH5α cells (New England Biolabs) using 4 tubes of cells. For each tube, 1 mL of outgrowth medium were added at 37° C. with shaking for 1 hr, then 9 mL of LB mixed with kanamycin, and shaken 250 rpm at 37° C. for overnight. A small aliquot was taken to plate for calculating titer. The next day, the cultures were pooled, and 4 mL of culture were frozen as phage tail library in 15 glycerol and stored at −80 (C. The rest of them were spun down for DNA extraction. Purified DNA (1 μg) was electroporated into freshly prepare Kp294 without glycerol. Ten tubes of freshly prepared cells were used, pooled and plated onto a large LB agar plate with kanamycin (Corning). Small aliquots with serial dilutions were also plated onto small plates. Plates were incubated in a 37° C. incubator overnight. The next day transformed Kp294 were scraped into LB supplemented with kanamycin and 15% glycerol and frozen at −80° C. Four independent tail libraries were constructed that varied in BC, EF and FG1 and FG2 loops separately (see FIG. 3 and Table 2 for these loops).

TABLE 1
Primer sequences for constructing pKan φ115
tail fiber (TF) into pKan28 backbone, TF loop
variants, and sequencing TF recombinant clones.
K represents guanine or thymine.

| Name | | | Tm |
|---|---|---|---|
| of oligo | Sequence 5′ | length | (° C.) |
| Kp φ115TF | GTCCCATTCGCCAatgac | 34 | 68 |
| F1 | taatatcaaggcccgc | | |
| | (SEQ ID NO: 4) | | |
| Kp φ115TF | GCCTATGGAAAAACctag | 40 | 61 |
| Rev | actactattctatttcct | | |
| | tttc | | |
| | (SEQ ID NO: 5) | | |
| pKan115TF | CACTGGATCATGGTAAGT | 36 | 68 |
| F1 | TTTTCCATAGGCTCCGCC | | |
| | (SEQ ID NO: 6) | | |
| pKan115TF | ttgatattagtcatTGGC | 32 | 67 |
| Rev | GAATGGGACGCGCC | | |
| | (SEQ ID NO: 7) | | |
| Kpφ115TF | GTGTAGCGGTCACGCTGC | 18 | 60 |
| seq F1 | (SEQ ID NO: 8) | | |
| Kp φ115TF | AGGAGCATCGATACGAAC | 22 | 60 |
| seq F5 | CTGA | | |
| | (SEQ ID NO: 9) | | |
| Kpφ115 TF | TTATAGTCCTGTCGGGTT | 22 | 59 |
| seq R1 | TCGC | | |
| | (SEQ ID NO: 10) | | |
| Kp φ115TF | CTGTATGCCGATATGTAT | 28 | 62 |
| seq R4 | GCCCTCTCTC | | |
| | (SEQ ID NO: 11) | | |
| Kp φ115TF | ATGTATGCCCTCTCTCTG | 28 | 60 |
| Fseq R5 | ATAAAACCAG | | |
| | (SEQ ID NO: 12) | | |
| Kpφ115TF | ggaagggggaaatagaat | 72 | 69 |
| BClp F1 | tctacagtatagacNNKN | | |
| | NKNNKNNKNNKggtacaa | | |
| | gagcatacgcgctacgtg | | |
| | (SEQ ID NO: 13) | | |
| Kpφ115 TF | gtctatactgtagaattc | 32 | 60 |
| BClp R1 | tatttcccccttcc | | |
| | (SEQ ID NO: 14) | | |
| Kpφ115TF | aagagaataggatcatcg | 64 | 68 |
| EFlp F1 | ttagggtaNNKNNKNNKN | | |
| | NKNNKNNKagcagcataa | | |
| | aaacattcac | | |
| | (SEQ ID NO: 15) | | |
| Kpφ115TF | taccctaacgatgatcct | 30 | 62 |
| EFlp R1 | attctcttgccc | | |
| | (SEQ ID NO: 116) | | |
| Kpφ115TF | agcagcataaaaacattc | 72 | 70 |
| FGlp1F1 | actccaNNKNNKNNKNNK | | |
| | NNKNNKNNKNNKNNKgga | | |
| | caagaaacgtctgatgga | | |
| | gtc | | |
| | (SEQ ID NO: 17) | | |

Approximately 50 μL of these frozen independent phage tail libraries (as described immediately above) were inoculated into 70 mL of LB mixed with kanamycin and grown into log phase (0.5-0.7 at A600). 10 mL of the log phase cells were then infected with φ115 stocks at multiple of infection (moi) 0.1 for 3 hrs. 400 μL of ChCl₃ were added into the culture and cells were spun down to collect the supernatant. Phages were tittered on various Klebsiella spp.

For sequencing, PCR fragments (354 bp) were amplified using synthetic phages in phage libraries as DNA templates and primers, Kpφ115TF seq F5 and Kpφ115TF seq R4. The resulting DNA fragments were separated by running 1% agarose running under 1× Tris Acetate-EDTA buffer. The 354 bp DNA fragments were then purified by QIAQUICK gel purification kit followed by QIAQUICK PCR purification kit (Qiagen). Approximately 2 μg of purified DNA was sent out for commercial Next Generation Sequencing (Genewiz) to confirm variations in loop sequences found within the phage libraries. Selected sequences appear below in Table 2. The first row in Table 2 (SEQ ID Nos: 18, 89, 124, and 176) represents the wild-type phage sequence. The columns in Table 2 should be considered as independent from one another, i.e., the sequence at one position in a row is not necessarily related to the sequences in the other positions (columns) in the same row.

TABLE 2
Variants in loop sequences found in
synthetic phage libraries as compared to
wild-type sequences (top row).

| BC loop | EF loop | FG1 loop | FG2 loop |
|---|---|---|---|
| TGNDG (SEQ ID NO: 18) | TQSGRG | WVERDNGTT | QETSDGVIYN |
| | (SEQ ID | (SEQ ID NO: 124) | (SEQ ID NO: 176) |
| | NO: 89) | | |
| NGNDG (SEQ ID NO: 19) | PQSGRG | WVERDNGNT | QETPDGVIYN |
| | (SEQ ID | (SEQ ID NO: 125) | (SEQ ID NO: 177) |
| | NO: 90) | | |
| TGNAG (SEQ ID NO: 20) | TQNGRG | WVERYNGTT | QETSDGVLYN |
| | (SEQ ID | (SEQ ID NO: 126) | (SEQ ID NO: 178) |
| | NO: 91) | | |
| TGNNG (SEQ ID NO: 21) | TQRGRG | WVARDNGTT | QETHDGVIYN |
| | (SEQ ID | (SEQ ID NO: 127) | (SEQ ID NO: 179) |
| | NO: 92) | | |
| TGDDG (SEQ ID NO: 22) | TPSGRG | WVERDNETT | QETSAGVIYN |
| | (SEQ ID | (SEQ ID NO: 128) | (SEQ ID NO: 180) |
| | NO: 93) | | |
| TGTDG (SEQ ID NO: 23) | AQSGRG | WVGRDNGTT | HETSDGVIYN |
| | (SEQ ID | (SEQ ID NO: 129) | (SEQ ID NO: 181) |
| | NO: 94) | | |
| TENDG (SEQ ID NO: 24) | GQPILR | FKVAQLCQV | KGCVGCLWVV |
| | (SEQ ID | (SEQ ID NO: 130) | (SEQ ID NO: 182) |
| | NO: 95) | | |
| AGNDG (SEQ ID NO: 25) | NQSGRG | GVERGNGTT | LDRSLCLRGS |
| | (SEQ ID | (SEQ ID NO: 131) | (SEQ ID NO: 183) |
| | NO: 96) | | |
| TGNEG (SEQ ID NO: 26) | SQSGRG | LVFAGDGSL | QDPSDGVIYN |
| | (SEQ ID | (SEQ ID NO: 132) | (SEQ ID NO: 184) |
| | NO: 97) | | |
| TGSDG (SEQ ID NO: 27) | SRKALE | RRVKVFLLT | QEKPDGVIYN |
| | (SEQ ID | (SEQ ID NO: 133) | (SEQ ID NO: 185) |
| | NO: 98) | | |
| TGHDG (SEQ ID NO: 28) | TCFVLF | RVERDNGTT | QEMSDGVIYN |
| | (SEQ ID | (SEQ ID NO: 134) | (SEQ ID NO: 186) |
| | NO: 99) | | |
| PGNDG (SEQ ID NO: 29) | TESGRG | SGAVPSTWG | QEPSDGVIYN |
| | (SEQ ID | (SEQ ID NO: 135) | (SEQ ID NO: 187) |
| | NO: 100) | | |
| TGNDE (SEQ ID NO: 30) | THSGRG | WAERDNGTT | QEPSNGVIYN |
| | (SEQ ID | (SEQ ID NO: 136) | (SEQ ID NO: 188) |
| | NO: 101) | | |
| TRNDG (SEQ ID NO: 31) | TQSGGG | WGERDNGTT | QETSDGAIYN |
| | (SEQ ID | (SEQ ID NO: 137) | (SEQ ID NO: 189) |
| | NO: 102) | | |
| RRPGL (SEQ ID NO: 32) | TQSGKG | WIERDNGTT | QETSDGVICN |
| | (SEQ ID | (SEQ ID NO: 138) | (SEQ ID NO: 190) |
| | NO: 103) | | |
| TGNDR (SEQ ID NO: 33) | TQSGMG | WVEGDNGTT | QETSDGVIYH |
| | (SEQ ID | (SEQ ID NO: 139) | (SEQ ID NO: 191) |
| | NO: 104) | | |
| NRNDG (SEQ ID NO: 34) | TQSGSG | WVERDHGTT | QETSDGVIYK |
| | (SEQ ID | (SEQ ID NO: 140) | (SEQ ID NO: 192) |
| | NO: 105) | | |
| PGHDG (SEQ ID NO: 35) | TQSRRG | WVERDNGAN | QETSDGVIYT |
| | (SEQ ID | (SEQ ID NO: 141) | (SEQ ID NO: 193) |
| | NO: 106) | | |
| SGNDG (SEQ ID NO: 36) | TQSWRG | WVERDNGTN | QETSDGVVYN |
| | (SEQ ID | (SEQ ID NO: 142) | (SEQ ID NO: 194) |
| | NO: 107) | | |
| TGNDV (SEQ ID NO: 37) | TRSGRG | WVERDNRTT | QETSNGVIYN |
| | (SEQ ID | (SEQ ID NO: 143) | (SEQ ID NO: 195) |
| | NO: 108) | | |
| IGNDG (SEQ ID NO: 38) | TKSGRN | WVERGNGTT | QETYDGVIYN |
| | (SEQ ID | (SEQ ID NO: 144) | (SEQ ID NO: 196) |
| | NO: 109) | | |
| TVNDG (SEQ ID NO: 39) | TQSGRD | WVESDNGTT | QGTSDGVIYN |
| | (SEQ ID | (SEQ ID NO: 145) | (SEQ ID NO: 197) |
| | NO: 110) | | |
| TCNDG (SEQ ID NO: 40) | TQSGRS | WVEREIGRA | QKTSDGVIYN |
| | (SEQ ID | (SEQ ID NO: 146) | (SEQ ID NO: 198) |
| | NO: 111) | | |
| TGKDG (SEQ ID NO: 41) | KIGRAH | DRKSTRLNS | RIRLLLSVRF |
| | (SEQ ID | (SEQ ID NO: 147) | (SEQ ID NO: 199) |
| | NO: 112) | | |
| TGNYR (SEQ ID NO: 42) | RSEEHT | GVERDNGTT | RTMFMQTCVS |
| | (SEQ ID | (SEQ ID NO: 148) | (SEQ ID NO: 200) |
| | NO: 113) | | |
| TGTEG (SEQ ID NO: 43) | RSEERR | LRDRRSLVV | RVRQTWPGSG |
| | (SEQ ID | (SEQ ID NO: 149) | (SEQ ID NO: 201) |
| | NO: 114) | | |
| TRNNG (SEQ ID NO: 44) | TDRKST | RSEEHKSEL | RWFPGESEVC |
| | (SEQ ID | (SEQ ID NO: 150) | (SEQ ID NO: 202) |
| | NO: 115) | | |
| AGHDG (SEQ ID NO: 45) | TQIGRA | RSEERRVGK | SGCTRIKSVS |
| | (SEQ ID | (SEQ ID NO: 151) | (SEQ ID NO: 203) |
| | NO: 116) | | |
| AGNDR (SEQ ID NO: 46) | TQRDRK | WRSEEHTSE | SWFSRLPLVG |
| | (SEQ ID | (SEQ ID NO: 152) | (SEQ ID NO: 204) |
| | NO: 117) | | |
| CRPGL (SEQ ID NO: 47) | TQSEIG | WRSEERRVG | TGSTIHVMNS |
| | (SEQ ID | (SEQ ID NO: 153) | (SEQ ID NO: 205) |
| | NO: 118) | | |
| NGNAG (SEQ ID NO: 48) | TQSGDR | WVDRKSTRL | VYRESSGSKV |
| | (SEQ ID | (SEQ ID NO: 154) | (SEQ ID NO: 206) |
| | NO: 119) | | |
| NGNGG (SEQ ID NO: 49) | TQSGKI | WVEIGRASC | WTLKLWVVML |
| | (SEQ ID | (SEQ ID NO: 155) | (SEQ ID NO: 207) |
| | NO: 120) | | |
| NGTDG (SEQ ID NO: 50) | TQSGRE | WVEKDNGTT | LTGSEVWIVK |
| | (SEQ ID | (SEQ ID NO: 156) | (SEQ ID NO: 208) |
| | NO: 121) | | |
| NRTEV (SEQ ID NO: 51) | TQSGRR | WVEKIGRAH | PLASFWRSSL |
| | (SEQ ID | (SEQ ID NO: 157) | (SEQ ID NO: 209) |
| | NO: 122) | | |
| NVNDG (SEQ ID NO: 52) | TQSGRV | WVEKIGRAS | QDRKSTRRTP |
| | (SEQ ID | (SEQ ID NO: 158) | (SEQ ID NO: 210) |
| | NO: 123) | | |
| PGDDG (SEQ ID NO: 53) | | WVERDKDRK | QDTPYGVIYN |
| | | (SEQ ID NO: 159) | (SEQ ID NO: 211) |
| PGNDE (SEQ ID NO: 54) | | WVERDKIGR | QEDRKSVVEG |
| | | (SEQ ID NO: 160) | (SEQ ID NO: 212) |
| PGRAG (SEQ ID NO: 55) | | WVERDNEIG | QETPAGVIYN |
| | | (SEQ ID NO: 161) | (SEQ ID NO: 213) |
| PGTDG (SEQ ID NO: 56) | | WVERDNGAT | QETPDGAIYN |
| | | (SEQ ID NO: 162) | (SEQ ID NO: 214) |
| TENEG (SEQ ID NO: 57) | | WVERDNGDR | QETPDGVIYK |
| | | (SEQ ID NO: 163) | (SEQ ID NO: 215) |
| TGDAG (SEQ ID NO: 58) | | WVERDNGKI | QETPDGVIYR |
| | | (SEQ ID NO: 164) | (SEQ ID NO: 216) |
| TGHAG (SEQ ID NO: 59) | | WVERDNGTR | QETPNGVIYN |
| | | (SEQ ID NO: 165) | (SEQ ID NO: 217) |
| TGNAR (SEQ ID NO: 60) | | WVERDNRSE | QETSDGDRKS |
| | | (SEQ ID NO: 166) | (SEQ ID NO: 218) |
| TGNGG (SEQ ID NO: 61) | | WVERDRKST | QETSDGVDRK |
| | | (SEQ ID NO: 167) | (SEQ ID NO: 219) |
| TGNYG (SEQ ID NO: 62) | | WVERDRKSV | QETSDGVIRS |
| | | (SEQ ID NO: 168) | (SEQ ID NO: 220) |
| TGTDR (SEQ ID NO: 63) | | WVERDRSEE | QETSDGVIYS |
| | | (SEQ ID NO: 169) | (SEQ ID NO: 221) |
| TRDDG (SEQ ID NO: 64) | | WVEREDRKS | QETSDRVIYN |
| | | (SEQ ID NO: 170) | (SEQ ID NO: 222) |
| TRHDG (SEQ ID NO: 65) | | WVERRSEEH | QETSRSEEHT |
| | | (SEQ ID NO: 171) | (SEQ ID NO: 223) |
| TRNDE (SEQ ID NO: 66) | | WVERRSEER | QETYDGVVYN |
| | | (SEQ ID NO: 172) | (SEQ ID NO: 224) |
| TVTDV (SEQ ID NO: 67) | | WVERSEEHT | QIGRASCRER |
| | | (SEQ ID NO: 173) | (SEQ ID NO: 225) |
| VINVS (SEQ ID NO: 68) | | WVRSEEHTS | |
| | | (SEQ ID NO: 174) | |
| TGHDV (SEQ ID NO: 69) | | WVXRDNGTT | |
| | | (SEQ ID NO: 175) | |
| TGNRS (SEQ ID NO: 70) | | | |
| AGDDG (SEQ ID NO: 71) | | | |
| PGNER (SEQ ID NO: 72) | | | |
| RSEER (SEQ ID NO: 73) | | | |
| TDRKS (SEQ ID NO: 74) | | | |
| TEIGR (SEQ ID NO: 75) | | | |
| TEMTE (SEQ ID NO: 76) | | | |
| TETDG (SEQ ID NO: 77) | | | |
| TETNG (SEQ ID NO: 78) | | | |
| TGDRK (SEQ ID NO: 79) | | | |
| TGKDR (SEQ ID NO: 80) | | | |
| TGNDK (SEQ ID NO: 81) | | | |
| TGNED (SEQ ID NO: 82) | | | |
| TGNEI (SEQ ID NO: 83) | | | |
| TGPDG (SEQ ID NO: 84) | | | |
| TGRSE (SEQ ID NO: 85) | | | |
| TRNAG (SEQ ID NO: 86) | | | |
| TRSEE (SEQ ID NO: 87) | | | |
| TRTDG (SEQ ID NO: 88) | | | |

Exemplary phage tail fiber proteins sequences are SEQ ID NOs: 226, 227, and 228 which are those of the phages Kpφ100, Kpφ115, and Kpφ111, respectively. FIG. 2 shows the predicted structure for the C-terminal-most 200 amino acid residues (C-200) of Klebsiella phage tail fiber and identified loop domains in two different orientations. The mutations described in Table 2 could be made other phages possessing a similar structure. The C-200 region is believed to be most critical for host determination.

FIG. 3 depicts an alignment of the C-terminal-most 200 amino acid residues in the tail regions of Kpφ115 and Kpφ111. One can see the lettered regions between which lie the highlighted loop regions where the synthetic phage described herein have mutations. These are the loops identified in Table 2 above (BC, EF, FG1, and FG2). The complete sequences of the two phage tail proteins shown, which include the C-200 regions of FIG. 3, are provided in SEQ ID NOs: 227 and 228, respectively.

### Phage Activity

The synthetic phage libraries were characterized using spot tests and plaque assays with various Klebsiella pneumoniae strains. For spot tests, Kp hosts were grown to log phase (A600=0.4-0.6) first and mixed with LB (M) top agar (0.6%) to create bacterial lawns. Phage samples were serially diluted, and 10 μL of each dilution was carefully spotted onto the solidified top agar layer, which was overlaid on either LB (M) or TS plates. Following incubation, individual plaques that formed within each spot were counted. The number of plaques was then used to calculate the overall titer for each tested phage, expressed as plaque-forming units per milliliter (PFU/mL), taking into account the dilution factor and the spotted volume.

As a measure of phage resistance, an OMNILOG liquid lysis assay was used to measure the respiration rate among the infected Kp and control without infection. In brief, bacteriophage growth was monitored in OMNILOG system with the respiration rate, in which the Kp was growth in tryptic-soy broth (TS) supplemented with 1% tetrazolium redox dye and challenged by the phages. The plate incubator recorded well color every 15 minutes as respiration of actively growing bacteria reduce the dye and generate a quantitative purple signal. Successful phage infection is measured by low or absent of color change due to suppressed bacterial respiration. The bacterial growth was measured over a 24-hour period.

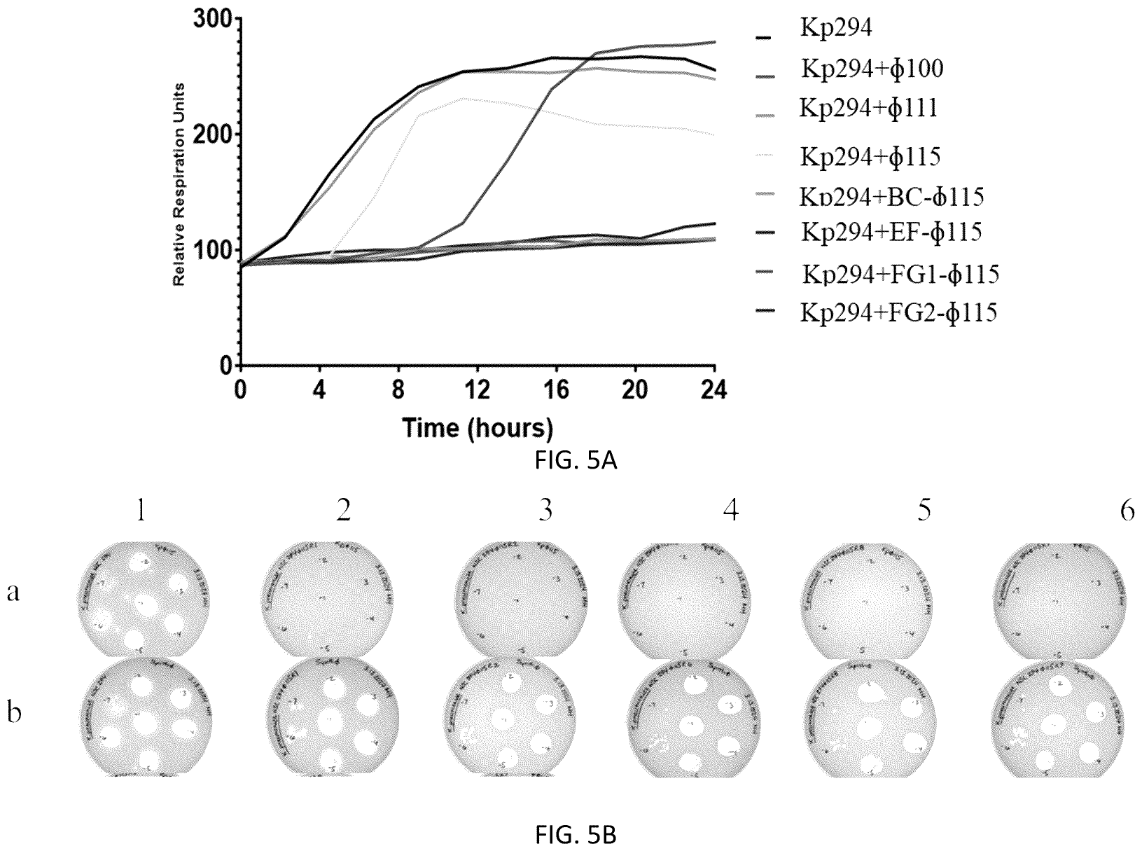

Low levels of bacterial respiration indicate successful bacterial lysis, while high respiration levels suggest continued bacterial growth. As expected, Kp294 alone and Kp294 infected with the non-lytic phage KpΦ111, a natural phage lysed Kp159, exhibited high respiration rates, similar to the uninfected control (FIG. 5A). In contrast, Kp294 infected with the four sub-synthetic phage libraries displayed low to no respiration (FIG. 5A), indicating effective bacterial lysis. Consistent with the respiration data, the sub-synthetic phage libraries produced clear zones of lysis on lawns of six phage-resistant Kp294 derivative strains (Kp294R) (FIG. 5B). The parental Φ115-1 and Φ115-2, however, did not produce visible plaques on these resistant strains, highlighting the enhanced infectivity of the synthetic phage libraries. Importantly, the synthetic phages also successfully infected and lysed the parental Kp294 strain.

A Kp multidrug resistance diversity panel (see Martin M J et al. “A panel of diverse Klebsiella pneumoniae clinical isolates for research and development.” Microb Genom 9(5):mgen000967 (2023)) of 96 distinct drug-resistance strains was used for testing the lysis assay. A 96-well plate was inoculated with the distinct Klebsiella strains. Bacterial cultures were grown overnight in LB media under shaking conditions at 37° C. Following incubation, the bacteria were diluted at a ratio of 1:30 by combining 5 μL of Klebsiella culture with 145 μL of LB media per well. Initial optical density (OD) measurements at 600 nm (A600) were recorded for each well to establish baseline growth. The plate was then incubated with shaking for 1 hour at 37° C. to allow bacteria to enter exponential growth phase. After the initial growth period, phages were added to each well and A600 measurements were immediately recorded to document the starting point for potential lysis. The plate was returned to the 37° C. shaking incubator for continued growth. Subsequent A600 measurements were taken at 1-hour intervals for all wells until the lysis process was complete, as indicated by stabilization of OD readings.

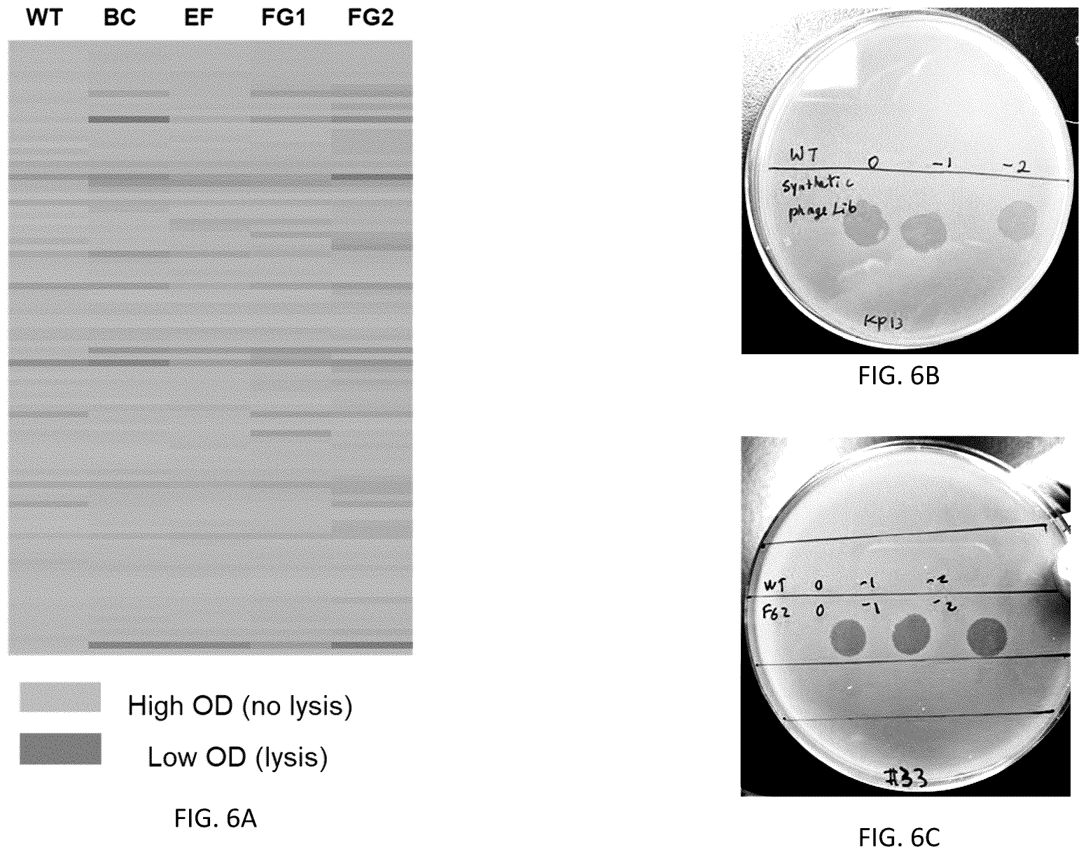

To assess the lytic activity of the synthetic phage libraries against the panel of drug-resistant Kp strains, a liquid lysis assay under standardized conditions was used. This approach allowed for efficient screening of phage activity against multiple strains simultaneously. A lysis heat map comparing the initial Kpφ115-1 stock (containing a low titer of φ100) to the four synthetic phage sub-libraries revealed a broader host range for the engineered phages (FIG. 6A). The synthetic libraries consistently lysed a greater number of drug-resistant Kp strains compared to the parental phage mixture. These findings were further corroborated by spot tests using two clinical isolates from the MRSN panel, Kp13 (MRSN 13761) and Kp33 (MRSN 25616). While the parental phage mixture failed to lyse these strains, the FG1 synthetic phage sub-library produced visible plaques (FIG. 6B), demonstrating its enhanced lytic capability.

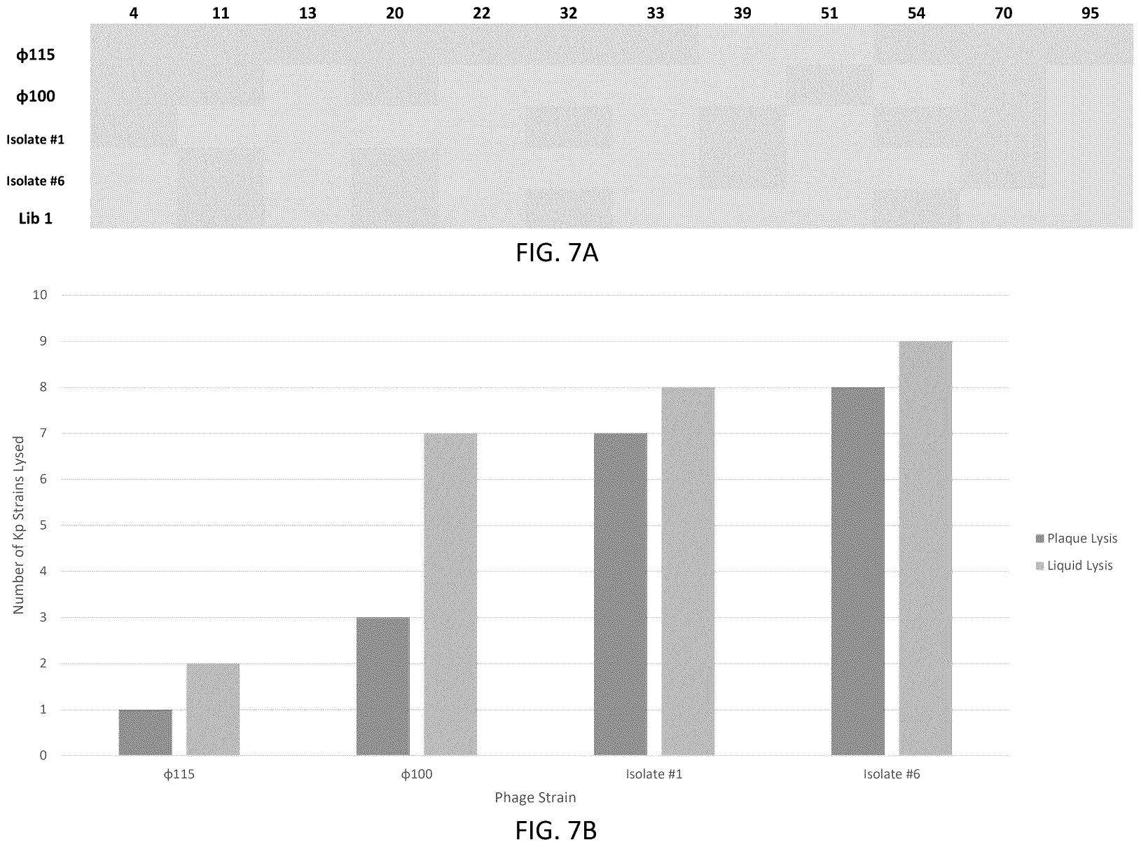

Host range analysis was performed on sixteen individual phages isolated from synthetic phage library Lib 1, following three rounds of enrichment on Kp strains, Kp13 and Kp294R1. Nine isolates were selected for further analysis via multiplex liquid lysis assays, alongside parental phages φ115, φ100, Lib 1, and Lib 2. The profiles for two isolates, #1 and #6 and Lib 1, φ115 and φ100 were listed in FIG. 7A. Isolate #6 exhibited the broadest host range, lysing 10 MRSN Kp strains in liquid culture. Subsequent spot assays confirmed plaque formation by isolate #6 on 8 of these strains (FIG. 7B). While the parental phage φ115-1 lysed 2 strains in liquid culture and only 1 on plates, φ100 demonstrated activity against 7 and 3 strains in liquid and spot assays, respectively. Notably, all tested individual isolates displayed an extended host range compared to both φ100 and φ115-1 when challenged against a panel of 96 multi-drug resistant Kp stains.

To determine the number of phages produced per infected bacterium, burst size analysis was performed using Klebsiella pneumoniae strains Kp294, Kp303, and Kp294R1 with the following phages: Kpφ115, Kpφ100, isolate #6, and isolate #1. The procedure began by mixing 11 mL of LB medium with 110 μL of an overnight culture of bacteria in a 50 mL conical tube flask (designated as culture 1). This mixture was incubated at 37° C. with shaking at 250 rpm until the optical density reached 0.25. At this point, 1.00×10⁷ bacteriophages were added to 10 mL of log phase cultures (multiplicity of infection=0.005), and timing was initiated. After 5 minutes of incubation at 37° C. without shaking, 100 μL of culture 1 was transferred to a fresh 10 mL of pre-warmed (37° C.) LB medium (designated as culture 2). At 5.5 minutes post-infection, 1 mL from culture 2 was transferred into a 1.5 mL centrifuge tube, and a 20 μL sample was quickly transferred into a PCR tube for Titer 1 measurement (non-adsorbed phages). Subsequently, 50 μL of chloroform was added to the centrifuge tube and vortexed, after which a 20 μL sample was transferred into another PCR tube for Titer 2 measurement. Following 30 minutes of incubation, during which cell lysis occurred resulting in only free phages, 1 mL from culture 2 was transferred into a 1.5 mL tube. After adding 50 μL of chloroform and vortexing, a 20 μL sample was transferred into a PCR tube for Titer 3 measurement. The number of initially infected cells (Ni) was calculated as Titer 2 minus Titer 1, and the burst size was determined by dividing Titer 3 by Ni. Two biological replicates were measured for standard error (SD) and two-sided t-test were used for measuring probability of difference (p<0.005 considered as significant difference).

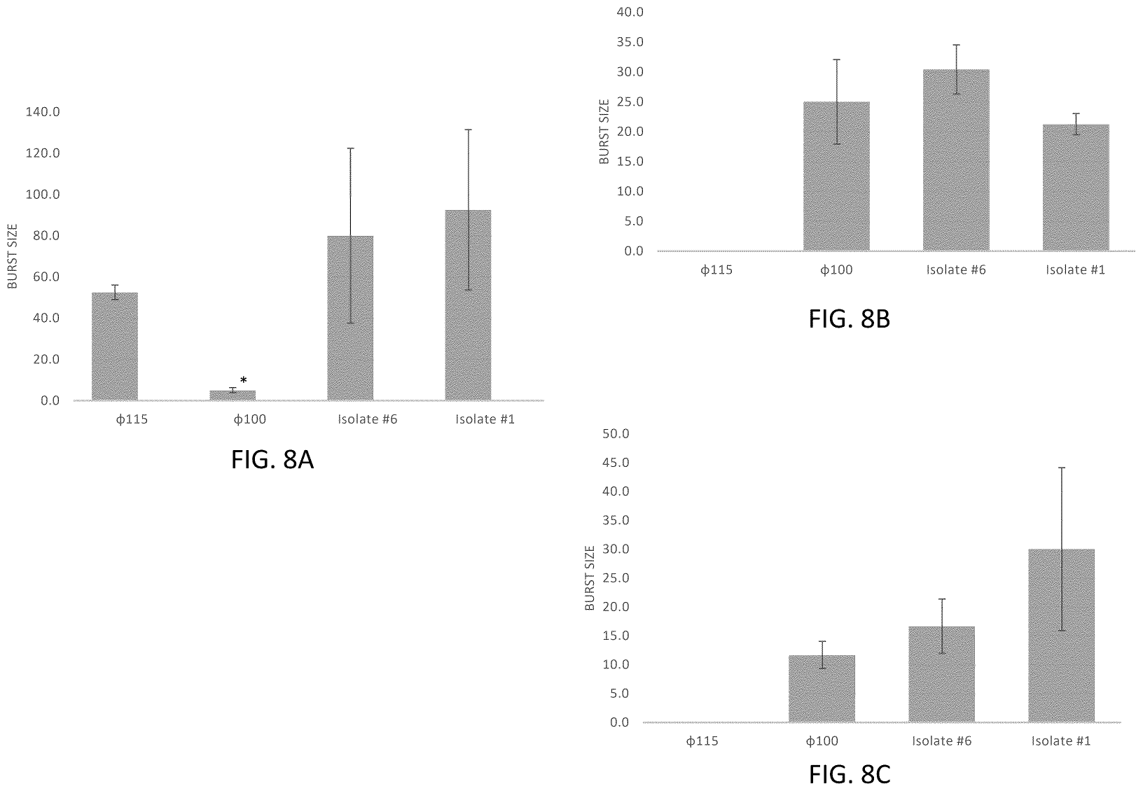

Burst size measurements were conducted according to the phage propagation protocol described above. While φ115-3 (phage stock developed from a single plaque growing on Kp294, but not on Kp303) exhibited a burst size of approximately 50 PFU per bacterium in Kp294, φ100 had a significantly smaller burst size (p<0.005) (FIG. 8A). In contrast, both isolate #6 and isolate #1 were not significantly different from φ115 (p>0.005). φ115-3 failed to produce detectable titers on strains Kp303 and Kp294R1, but the burst size for isolate #6 and isolate #1 were not significantly different from φ100 (p>0.005 in Kp303 and Kp294R1) (FIGS. 8B and 8C). Interestingly, isolate #1 exhibited a considerably larger burst size (30 PFU/CFU) compared to both φ100 and isolate #6 in strain Kp294R1, though still not significantly (p>0.005 listed in FIG. 8C).)

### Individual Phage Sequencing

To isolate individual phage strain from a mixed library, a three-round purification protocol was implemented. In the first round, the phage library was plated on a bacterial lawn, and a single, well-isolated plaque was selected and picked using a sterile pipette tip. This plaque was then used to inoculate an exponential growth culture of the host bacteria, followed by incubation at 37° C. until complete lysis was observed. The lysate was filtered to remove bacterial debris, and the resulting filtrate was used for a second round of plating. From this second plate, another single plaque was selected, and the process of liquid culture growth and filtration was repeated. A third and final round of plaque isolation, growth, and filtration was performed to ensure the resulting phage preparation contained only a single phage strain. This rigorous three-round isolation procedure effectively eliminated contaminating phages, yielding a genetically homogeneous phage isolate for subsequent characterization.

A phenol-chloroform DNA extraction was used to obtain phage genomic DNA. Phages were triple plaque-purified, then each isolate was amplified at 37° C. by infecting a 100 mL culture of Kp host until complete lysis occurred, then DNA extraction was performed on the lysate. Lysates were passed through a 0.22 μm filter and treated with DNAseI plus MgCl₂ to digest un-encapsulated DNA. Capsids were then disrupted with proteinaseK and SDS, and the genomes were extracted twice with phenol-chloroform-isoamyl alcohol. After debris and phage removal, genomic DNA was subsequently precipitated with PEG/NaCl, washed in 80% ethanol, resuspended in water, subjected to RNAse A digestion, re-extracted, ethanol-precipitated, and finally washed in 70% ethanol before resuspended in sterile water. Quality control was performed via agarose-gel confirmation of high molecular weight DNA, Qubit fluorimetry for concentration, and NanoDrop spectrophotometry for purity, before storage at 4° C. (−20° C. for long-term storage).

Isolated phage sequences are provided in SEQ ID NOs: 229 to 225. Table 3 below references these sequence identification numbers against the isolate numbers referred to herein.

TABLE 3
Cross-reference of isolate numbers and SEQ ID NOs.

| Synthetic phage isolate number | SEQ ID NO: |
|---|---|
| 1 | 230 |
| 6 | 231 |
| 7 | 232 |
| 8 | 233 |
| 13 | 235 |
| 14 | 236 |
| 17 | 237 |
| 18 | 238 |

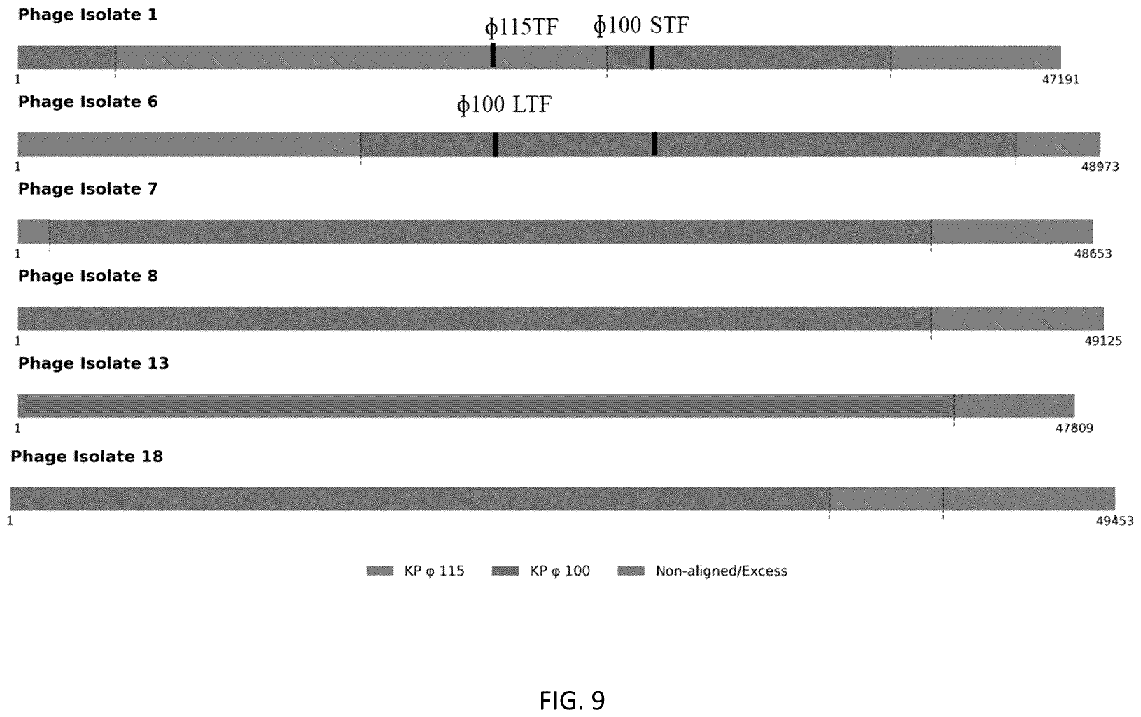

Genomic DNA sequencing revealed that each isolate harbored segments from both the Kpφ100 and φ115 genomes, albeit at varying ratios and locations (FIG. 9). Notably, isolates derived from Kp294R1 (phage isolate 13 and 18 exhibited a predominance of the φ100 genome, including both long tail fiber (LTF) and short tail fiber (STF) genes. In contrast, isolates (#1, #6) originating from Kp13 displayed a higher proportion of the φ115. Interestingly, isolates #1 and #6, while both possessing the φ115 major capsid genes, differed in their tail fiber composition: #1 contained the φ115 LTF and φ100 STF, whereas #6 harbored both φ100 tail fibers. Isolates #7, #8, #13 and #18 were characterized by a majority of φ100 genomic DNA (FIG. 9).

### Further Embodiments

Contemplated herein are phages and libraries thereof created against other bacteria besides the Klebsiella as described above. These can be developed using machine learning and structure prediction modeling to identify a likely receptor binding domain, then introducing random mutations therein and recombining them into the phage genomes to develop diverse synthetic phages.

Also contemplated herein is treatment with phages and libraries thereof produced as described. A method for treating or preventing infection in a subject in need thereof (for example a mammal such as a human) can include administering to the subject a composition comprising at least one bacteriophage as described herein and a pharmaceutically acceptable carrier; wherein the composition is administered at a dose sufficient to reduce the level of the at least one bacterium in the subject. In various aspects, the infection is a Klebsiella infection.

### Advantages

This technique can be used to prepare synthetic diverse phages in a shorter time than a yeast platform. The method has the flexibility to make synthetic phages that target other bacteria species. Phages with a large genomic DNA can also be used as scaffolds to prepare synthetic phages.

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Kevin Yehl, Sébastien Lemire, Andrew C Yang, Hiroki Ando, Mark Mimee, Marcelo Der Torossian Torres, Cesar de la Fuente-Nunez, Timothy K Lu (2019) Engineering Phage Host-Range and Suppressing Bacterial Resistance through Phage Tail Fiber Mutagenesis. Cell, 179(2): 459-469.
2. Hiroki Ando, Sebastien Lemire, Diana P.Pires, Timothy K.Lu. (2015) Engineering Modular Viral Scaffolds for Targeted Bacterial Population Editing. Cell Systems, 1(3):187-196.
3. Ido Yosef, Moran G. Goren, Rea Globus, Shahar Molshanski-Mor, and Udi Qimron (2017) Extending the Host Range of Bacteriophage Particles for DNA Transduction. Molecular Cell 66, 721-728.
4. Pedregosa F, Varoquaux G, Gramfort A, Michel V, Thirion B, Grisel 0, et al. Scikit-learn: Machine Learning in Python. J Mach Learn Res. 2011; 12:2825-30.
5. Samuel Kilcher, Patrick Studer, Christina Muessner, Jochen Klumpp, and Martin J. Loessne (2018) Cross-genus rebooting of custom-made, syntheticbacteriophage genomes in L-form bacteria. PNAS 115(3):567-572.
6. Taslem Mourosi J, Awe A, Guo W, Batra H, Ganesh H, Wu X, Zhu J. Understanding Bacteriophage Tail Fiber Interaction with Host Surface Receptor: The Key “Blueprint” for Reprogramming Phage Host Range. Int J Mol Sci. 2022 Oct. 12; 23(20):12146. doi: 10.3390/ijms232012146. PMID: 36292999; PMCID: PMC9603124.

1. A bacteriophage comprising:
at least one mutation in a region selected from the group consisting of the BC loop, the EF loop, FG1 loop, and the FG2 loop,
wherein the at least one mutation comprises a protein sequence selected from the group consisting of SEQ ID NOs: 19 to 88 in the BC loop, SEQ ID NOs: 90 to 123 in the EF loop, SEQ ID NOs: 125 to 175 in the FG1 loop, and SEQ ID NOs: 177 to 225 in the FG2 loop.

2. The bacteriophage of claim 1, wherein said at least one mutation comprises two or more mutations in at least two of said loops comprise the mutations.

3. The bacteriophage of claim 1, wherein, prior to taking into account said one or more mutations, said bacteriophage comprises a wild-type protein sequence of SEQ ID NO: 227 or SEQ ID NO: 228.

4. The bacteriophage of claim 1, wherein said the bacteriophage is capable of infecting Klebsiella.

5. The bacteriophage of claim 1, having a genomic sequence comprising a nucleotide sequence at least 99% identical to a sequence selected from the group consisting of SEQ ID NOS: 229 to 255.

6. The bacteriophage of claim 5, wherein said sequence is SEQ ID NO: 230 or SEQ ID NO: 231.

7. A phage library comprising a plurality of bacteriophages according to claim 1.

8. The phage library of claim 7, further comprising at least a second phage of wild-type Φ100 stock with genomic DNA comprising a sequence at least 99% identical to SEQ ID NO: 3.

9. A phage library comprising:
at least one phage comprising a genomic sequence comprising a nucleotide sequence at least 99% identical to a sequence selected from the group consisting of SEQ ID NOS: 229 to 255.

10. The phage library of claim 9, further comprising at least a second phage of wild-type Φ100 stock with genomic DNA comprising a sequence at least 99% identical to SEQ ID NO: 3.

11. A nucleic acid comprising:
a sequence encoding at least a portion of the C-terminal-most 200 amino acid residues of a phage tail protein sequence,
wherein said at least a portion comprises at least one mutation in a region selected from the group consisting of the BC loop, the EF loop, FG1 loop, and the FG2 loop,
wherein the at least one mutation comprises a protein sequence selected from the group consisting of SEQ ID NOs: 19 to 88 in the BC loop, SEQ ID NOs: 90 to 123 in the EF loop, SEQ ID NOs: 125 to 175 in the FG1 loop, and SEQ ID NOs: 177 to 225 in the FG2 loop.

12. A phage comprising:
a protein sequence encoded by any one of SEQ ID NOs: 229 to 255 and variations thereof that encompass sequences of 99% or better identity.

13. A method of preparing a synthetic bacteriophage library, the method comprising:
providing a selection of bacteriophage sequences;
using gradient-boosting machine learning and structure prediction modeling to identify a likely receptor binding domain in the selection;
introducing random mutations in the likely receptor binding domain to produce modified phage sequences; and
recombining the modified phage sequences into phage genomes to develop diverse synthetic phages.

14. The method of claim 13, further comprising testing said synthetic phages against suitable bacteria to identify phages active against desired targets, wherein the desired targets comprise drug-resistant and/or phage-resistant bacteria.

15. The method of claim 13, wherein said diverse synthetic phages are effective to kill Klebsiella.

16. The method of claim 14, wherein said diverse synthetic phages are effective to kill Klebsiella.
